(19) United States
(12) Patent Application Publication
(10) Pub. No.: US 2024/0216265 A1
(43) Pub. Date: Jul. 4, 2024

(54) LIPOSOMAL VEGAN PROBIOTIC SKIN TREATMENT

(71) Applicant: Voesh Corporation, South Plainfield, NJ (US)

(72) Inventor: Vera Seunghee OH, South Plainfield, NJ (US)

(21) Appl. No.: 18/148,583

(22) Filed: Dec. 30, 2022

(51) Int. Cl. A61K 8/99; A61K 8/14; A61K 8/34; A61K 8/37; A61K 8/55; A61Q 19/00

(52) U.S. Cl. CPC A61K 8/99; A61K 8/14; A61K 8/345; A61K 8/375; A61K 8/553; A61Q 19/007; A61K 2800/30; A61K 2800/56; A61K 2800/85

(57) ABSTRACT

A liposomal vegan probiotic ingredient for addition to skin formulations includes a Lactobacillus, a polyol, a non-ionic surfactant, a phospholipid and an oil. In one embodiment, the Lactobacillus is fermented, and a liposome mixture is extracted from the same. The liposome mixture is then added to several other cosmetic mixtures composed of various components and then heated and extruded such that the liposomal vegan probiotic can be added to skin treatments.

### BACKGROUND

### Technical Field

The present invention relates to a cosmetic mixture composed of various components that can be used in skin treatment formulations such as, for example, moisturizers, sunscreens, makeup and lip balms. More particularly, it relates to liposomal vegan probiotic in the form of a cosmetic mixture composed of various components that can be used in various skin treatment formulations and which compound includes probiotic Lactobacillus Ferment.

### Description of the Prior Art

The skin epidermis, along with sweat and sebaceous glands provide a total skin surface of about 25 m². Skin aging has intrinsic (genetic) and extrinsic (environmental) components. The most marked age-related changes may occur on the face, neck, forearm, and dorsal hands. Since dry skin plays an important role in the formation of fine wrinkles, many people make an effort to moisturize their skin using cosmetics or dietary supplements.

Probiotics can alter the composition of the intestinal microbiome, produce antimicrobial substances, and stimulate the body's immune response. Lactobacillus is a type of bacterium that makes lactic acid through fermentation of carbohydrates. Lactobacillus is the largest genus of the family Lactobacilliae, and includes more than 200 published species, as of 2019. It is considered a good bacteria that is found in the human body. Since 1994, strains with similarity of more than 97% to the 16S rRNA gene sequence have been considered to belong to the same species. Species that are closely related, such as those belonging to the Lactobacillus plantarum group, including L. fabifermentans, L. plantarum, L. paraplantarum, and L. pentosus are indistinguishable using 16S rRNA gene sequencing because of the high degree of similarity (as high as 99%) of the 16S rRNA gene sequences among the species. Within one species, strains and subgroups can differ by the disease they produce, their environmental habitat, and many other characteristics. Correct identification of probiotic strains at the species level is important for safety assessment, as it allows a connection to potentially relevant, species-related scientific, and technological information.

“Liposomes” are a particular type of vesicle that are widely employed for biomedical and biotechnological purposes. The liposomes are composed of one or more closed shells, or lamellae, of a phospholipid bilayer that can enclose a small volume of aqueous or lipophilic liquid. Liposomes are promising nanocarriers, representing a biocompatible, safe, and efficient delivery system for both hydrophilic and lipophilic compounds. The liposomes can provide payload protection, transportation through biological membranes, and increased dosage delivered to a target.

### SUMMARY

In accordance with one implementation, the liposomal vegan probiotic formulation includes fermented Lactobacillus media in an amount in a range of 1×10⁹ to 1×10¹¹ CFU/g. Also included is a polyol in an amount of 1% to 20 wt %, a non-ionic surfactant in a range of 0.005% to 0.1 wt. %, a phospholipid in an amount of 0.000002% to 0.00004 wt %, and an oil.

In accordance with another implementation, the liposomal vegan probiotic formulation includes a fermented Lactobacillus media in an amount in a range of about 1×10⁹ to 1×10¹¹ CFU/g; a propanediol in an amount in a range of 1% to 20 wt. %, a 1,2-hexanediol in an amount in a range of 0.1% to 2 wt. %; and ethyl-hexyl-glycerin in an amount in a range of 0.005% to 0.1 wt. %. A caprylic/capric triglyceride in an amount in a range of 0.000002% to 0.00004 wt. %, hydrogenated lecithin in an amount in a range of 0.000001% to 0.00002 wt. %, polyglyceryl-6 caprylate in an amount in a range of 0.0000005% to 0.000001 wt. %; and polyglyceryl-4 caprate in an amount in a range of 0.0000005% to 0.000001 wt. %. with a balance water (H₂O).

In accordance with another implementation, a method of preparing a liposomal vegan probiotic formulation includes fermenting a Lactobacillus media composition, and then extracting a liposome mixture from the fermented Lactobacillus media composition. A propanediol is added in an amount in a range of 1% to 20%. 1,2-hexanediol is added in an amount in a range of 0.1% to 2%. Ethyl-hexyl-glycerin is added in an amount in a range of 0.005% to 0.1%. A caprylic/capric triglyceride is added in an amount in a range of 0.000002% to 0.00004%. Hydrogenated lecithin is added in an amount in a range of 0.000001% to 0.00002%. Polyglyceryl-6 caprylate is added in an amount in a range of 0.0000005% to 0.000001%, and Polyglyceryl-4 caprate is added in an amount in a range of 0.0000005% to 0.000001%. The mixture is heated to to 80° C. to 9° 0C. The mixture is then extruded at 300 rpm to 600 rpm in 5 minutes to 20 min using a microfluidizer at a pressure in a range of about 500 bar to about 1500 bar.

Other aspects and features of the present principles will become apparent from the following detailed description considered in conjunction with the accompanying drawings. It is to be understood, however, that the drawings are designed solely for purposes of illustration and not as a definition of the limits of the present principles, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings wherein like reference numerals denote similar components throughout the views:

FIG. 1 shows a block/flow diagram for a process of preparing the Lactobacillus plantarum formulation according to an embodiment of the invention; and

FIG. 2 shows an illustration of the fermenter and extruder portions of the process according to an embodiment of the invention.

### DETAILED DESCRIPTION

Principles and embodiments of the present invention relate to the use of probiotics for skin health and the ability of the probiotics to alter the composition of the skin cells to regulate protective mechanisms in the skin. Although intrinsic and extrinsic aging are triggered by different factors, both involve similar molecular mechanisms.

Principles and embodiments of the present invention relate to the microbiology and nutritional effects of a probiotic formulation based on Lactobacillus Plantarum.

Dry skin is caused by an imbalance between the amount of moisture in the stratum corneum and the intercellular lipids, which is a prominent clinical manifestation of the skin-aging process.

In various embodiments, the topical administration of a skin treatment including the probiotic cosmetic mixture composed of various components of the present invention exerts anti-photoaging effects through reduction of wrinkle formation and suppression of epidermal thickening and that skin hydration increases in association with increasing ceramide level via regulation of serine palmitoyltransferase and ceramidase expression, as shown in mice skin.

Embodiments of the present invention relate to the use of a Lactobacillus ferment as part of the cosmetic mixture composed of various components to ameliorate the effects of skin aging, such as improve skin hydration, wrinkles, skin gloss, and skin elasticity, due to extrinsic factors through the regulation of skin conditions. The embodiments of the present invention provide a bacterial composition which includes from 1×10⁹ to 1×10¹¹ CFU/g of Lactobacillus ferment cells which have the ability to significantly increase the skin moisture content and reduce signs of facial aging by limiting wrinkles and improving elasticity and skin gloss.

By way of one example of a Lactobacillus ferment strain of the present invention, Lactobacillus Plantarum JCM 1149 cells have been shown to increase ceramide level via regulation of serine palmitoyltransferase and ceramidase expression and ameliorate inflammation by reducing matrix metallopeptidases (MMP-2 and MMP-9), zonulin, and calprotectin in plasma, all of which are related to skin and intestinal permeability. Lactobacillus Plantarum JCM 1149 cells have been shown to upregulate IGFBP5, SERPINE1, EFEMP1, COL6A3, and SEMA3B and downregulate MT2A, MT1E, MT1X, MT1G, and MT1F between TNF-α and TNF-α plus HY7714 treated Caco-2 cells. Thus, embodiments of the bacterial composition of L. Plantarum JCM 1149 can be useful as a nutricosmetic agent for its anti-aging benefit to the skin.

Embodiments of the present invention relate to the use of a novel Lactobacillus ferment strain as part of a cosmetic mixture composed of various components that have improved skin wrinkle and moisturizing effects, and a product containing the same as an active ingredient. Embodiments of the present invention relate to the use of the probiotics of a novel Lactobacillus ferment strain as one ingredient in a compound of ingredients to form a probiotic cosmetic mixture composed of various components added to anti-wrinkle creams, anti-aging products and/or skin moisturizers for skin wrinkle inhibitory and moisturizing effects.

Aspects of the present principles are described with reference to flowchart illustrations and/or block diagrams of methods.

In one or more embodiments, the liposomal vegan probiotic formulation includes: a Lactobacillus Ferment, a polyol, a non-ionic surfactant, a phospholipid, and an oil. The units of measurement are in weights and/or weight percent (wt. %), for example, Oil 30 g, Active 20 g, Water 50 g=>Oil 30 wt. %., Active 20 wt. %, Water 50 wt. % Total is 100%.

In various embodiments, the oil used in the Liposome is MCT oil (Caprylic/Capric Triglyceride) which can control the liposome particle size by being covered into a hydrophobic part.

In various embodiments, the polyol can be selected from a group including, but not limited to, polyethylene glycol, glycerin, dipropylene, glycolbutylene glycol, pentylene glycol, isopentyldiol, hexanediol, methyl propanediol, sorbitol, diglycerin, erythritol, pentaerythritol, polybutylene glycol-10, ethyl-hexyl-glycerin, polyglycerin3, polyglycerin-4, polyglycerin-6, polyglycerin-10, polyglycerin-20, polyglycerin-40, sorbeth-5, sorbeth-6, sorbeth-20, sorbeth-30, sorbeth-40, inositol, maltitol, mannan, mannitol, mannose, lactitol, lactose, dihydroxy propyl PG-glucoside, dithiaoctanediol, fructose, glucamine, methyl glucamine, glucose, 1,2,6-hexanethiol, methyl gluceth10, methyl gluceth-20, ozonized glyceri, phytantriol, thioglycerin, threitol, trimethylolpropane, xylitol, and combinations thereof.

In various embodiments, the non-ionic surfactant can be selected from a group including: polyoxyethylene sorbitan fatty acid ester, for example, polysorbate 20, polysorbate 60, polysorbate 80; polyoxyethylene alkyl ether, for example, isoceteth 20, steareth 2, steareth 10, steareth 20, steareth 21, laureth 4, ceteareth 16, ceteareth 20, ceteth 2, ceteth 20; polyglyceryl fatty acid ester: polyglyceryl-4 caprate, polyglyceryl-10 caprate, polyglyceryl-10 laurate, polyglyceryl-10 dilaurate, polyglyceryl-10 oleate, polyglyceryl-10 dioleate, polyglyceryl-10 stearate, polyglyceryl-10 isostearate, polyglyceryl-10 disostearate, polyglyceryl-6 caprylate, polyglyceryl-6 ricinolate, polyglyceryl-5 laurate, polyglyceryl-5 dilaurate, polyglyceryl-5 myristate, polyglyceryl-5 trimyristate, polyglyceryl-5 oleate, polyglyceryl-5 dioleate, polyglyceryl-5 trioleate, polyglyceryl-5 steate, (polyglyceryl-5 tristeate, polyglyceryl-5 hexasteate, polyglyceryl-4 carprate, polyglyceryl-3 cocoate; and combinations thereof.

In various embodiments, the phospholipids can be selected from a group including: natural phospholipids, for example, phosphatidyl choline, lecithin, egg lecithin, soy lecithin, hydrogenated lecithin, sphingomyelin, cholesterol, phosphatidic acid, phosphatidyl serine, phosphatidyl glycerol, phosphatidyl inositol, phosphatidyl ethanolamine, diphosphatidyl glycerol, cardiolipin, and plasmalogen; synthetic phospholipids, for example, dicetyl phosphate, distearoyl phosphatidyl choline, dioleoyl phosphatidyl ethanolamine, dipalmitoyl phosphatidyl choline, dipalmitoyl phosphatidyl ethanolamine, dipalmitoyl phosphatidyl serine, eleostearoyl phosphatidyl choline, eleostearoyl phosphatidyl ethanolamine, eleostearoyl phosphatidyl serine; and combinations thereof.

In various embodiments, the oil can be selected from a group including: plant derived oil, for example, Helianthus Annuus (Sunflower) Seed Oil, Zea Mays (Corn) Oil), Glycine Max (Soybean) Oil, Persea Gratissima (Avocado) Oil, Sesamum Indicum (Sesame) Seed Oil Olea Europaea (Olive) Fruit Oil, caprylic/capric triglyceride, and combinations thereof.

Without oil (i.e., hydrophobic) parts, the liposome structure is unstable, and it will affect the size of the liposome, which will start becoming smaller.

In one or more embodiments, the liposomal vegan probiotic formulation include: a Lactobacillus Ferment, propanediol, 1,2-hexanediol, ethyl-hexyl-glycerin, caprylic/capric triglyceride, hydrogenated lecithin, polyglyceryl-6 caprylate, and polyglyceryl-4 caprate.

In one or more embodiments, the formulation is a liquid at room temperature (i.e., 25° C.). In various embodiments, the formulation can be applied to the surface of the skin of a person, as the liquid.

In various embodiments, the propanediol acts as a solvent to dissolve other components of the formulation.

In various embodiments, the 1,2-hexanediol acts as a solvent to dissolve other components of the formulation.

In various embodiments, the ethyl-hexyl-glycerin acts as a solvent to dissolve other components of the formulation.

In various embodiments, the caprylic/capric triglyceride acts as a binder.

In various embodiments, the hydrogenated lecithin acts as a phospholipid.

In various embodiments, the polyglyceryl-6 caprylate acts as an emulsifier to modify and/or enhance the viscosity.

In various embodiments, the polyglyceryl-4 caprate acts as a surfactant to help mix water-based and oil-based ingredients.

Liposomes have a lamella body structure similar to a cell membrane. This can allow the liposome to penetrate into the skin and thereby affect and improve a skin condition. In this liposomal structure, hydrophilic Lactobacillus ferments can be absorbed into the skin more effectively.

The term, “CFU” means colony forming unit, which is a unit commonly used to estimate the concentration of microorganisms in a test sample. The term “cfu/g” relates to the gram weight of the composition as such, including relevant additives present in the composition. The term “CFU/day” relates to a dosage of the number of colony forming units (CFUs) of the composition provided to a subject.

In a preferred embodiment, the Lactobacillus media composition is prepared and fermented, where the Lactobacillus Plantarum strain is isolated and grown in the lab from fermented cabbage.

In various embodiments, the Lactobacillus media composition can include:

Yeast extract 0.004 to about 8 wt. %, Soy peptone 0.01 to about 10 wt. %, Glucose 0.01 to about 20 wt. %, Ammonium citrate dibasic 0.002 to about 4 wt. %, Potassium phosphate dibasic 0.002 to about 4 wt. %, Sodium acetate anhydrous 0.003 to about 3 wt. %, Magnesium sulfate heptahydrate 0.0001 to about 0.3 wt. %, Manganese sulfate pentahydrate 0.00005 to about 0.1 wt. %.

In various embodiments, the Lactobacillus media composition can be fermented by stirring at about 140 rpm to about 200 rpm, at a fermentation temperature in a range of about 25° C. to about 45° C., for a fermentation time in a range of about 15 hours to about 36 hours to produce a fermented liposome suspension.

In various embodiments, the fermented liposome suspension can be passed through a membrane filter using and extruder equipped with a pump, which pushes fluids through the membrane(s). The membranes can have a defined pore size in a range of about 100 nm to about 300 nm. The extrusion can be done using a microfluidizer from 500 bar to 1,500 bar of pressure, where the liposome is pressed out at about 700 bar. The mechanism involves forcing a coarse emulsion through microchannels to a particular area by a pump by pressurizing compressed air up to about 150 MPa, which results in a nano-emulsion, where different numbers of passes lead to different sizes.

Heating and mixing with Propanediol of about 1 wt. % to about 20 wt. %, 1,2-Hexanediol about 0.1 wt. % to about 2 wt. %, Ethylhexylglycerin about 0.005 wt. % to about 0.1 wt. %, Caprylic/Capric Triglyceride about 0.000002 wt. % to about 0.00004 wt. %, Hydrogenated Lecithin of about 0.000001 wt. % to about 0.00002 wt. %, Polyglyceryl-6 Caprylate about 0.00000005 wt. % to about 0.000001 wt. %, Polyglyceryl-4 Caprate about 0.00000005 to about 0.000001 wt. %; at about 80° C. to about 90° C., and add to the Lactobacillus Ferment about 10 wt. % to about 90 wt. % by heating and mixing at about 30° C. to about 60° C., and at about 300 rpm to about 600 rpm for about 5 minutes (min) to about 20 min. By using a Microfluidizer, it can produce liposomes at about 500 bar to about 1500 bar on 1 to 5 Cycles (repeated passes through the microfluidizer).

In various embodiments, propanediol in an amount in a range of about 1 wt. % to about 20 wt. % can be added to the extruded liposome compound.

In various embodiments, ethyl-hexyl-glycerin in an amount in a range of about 0.005 wt. % to about 0.1 wt. % can be added to the extruded liposome compound.

In various embodiments, caprylic/capric triglyceride in an amount in a range of about 0.000002 wt. % to about 0.00004 wt. % can be added to the extruded liposome compound.

In various embodiments, hydrogenated lecithin in an amount in a range of 0.000001 wt. % to about 0.00002 wt. % can be added to the extruded liposome compound.

In various embodiments, polyglyceryl-6 caprylate in an amount in a range of about 0.00000005 wt. % to about 0.000001 wt. % can be added to the extruded liposome compound.

In various embodiments, 1,2-hexanediol in an amount in a range of about 0.1 wt. % to about 2 wt. % can be added to the extruded liposome compound. The ingredients can be added by pre-mixing the 1,2-hexanediol, propanediol, ethyl-hexyl-glycerin, caprylic/capric triglyceride, hydrogenated lecithin, polyglyceryl-6 caprylate, and polyglyceryl-4 caprate together in the predetermined amounts and introducing the mixture into the hooper of the microfluidizer. The mixture can be extruded at a volumetric rate in a range of about 5,000 to about 50,000 cubic centimeters a minute.

In various embodiments, polyglyceryl-4 caprate in an amount in a range of about 0.00000005 wt. % to about 0.000001 wt. % can be added to the extruded liposome compound.

Skin hydration was measured in the stratum corneum of the cheek (using the intersection of a vertical line from the corner of the eye and a horizontal line from the tip of the nose as the sampling point), the forearm, and the hand using a corneometer (CM825; Courage and Khazaka Electronic GmbH, Cologne, Germany). This device measures the variation in the electrostatic capacity, which is dependent on the moisture content in the stratum corneum. Measurements are reported in arbitrary units of the electrostatic capacity.

To evaluate the effect of the bacteria formulation on skin hydration, the water content of the face, forearm, and hand of subjects was first measured using the Corneometer CM825. The water content in both groups was significantly increased (p<0.001) from baseline at weeks 4, 8, and 12, and the rates of retention of water content in the face and hand were significantly higher in the probiotic group than in the placebo group (p<0.01, face; and <0.05, hand) at week 12.

Trans-epidermal water loss from the face, forearm, and hand were measured using a vapometer (SWL4001, Delfin, Finland). This device has a humidity sensor in a cylindrical measurement chamber that records changes in relative humidity (RH) inside the chamber during the measurement and automatically calculates trans-epidermal water loss (g/m²h).

The chamber is closed by the skin or other measurement surface during the measurement period and is unaffected by ambient airflows. The sensor monitors the increase of relative humidity (RH) inside the chamber during the measurement phase and the evaporation rate value (g/m²h) is automatically calculated from the RH increase. Ambient temperature and humidity are recorded using an external room sensor. All hydration and water loss measurements were performed three times at each point and the averages were used in the analysis.

There were significant increases in the skin water content in the face (p<0.01) and hands (p<0.05) after 12 weeks of use of the probiotic.

Water loss was significantly decreased from baseline in both groups at weeks 4, 8, and 12 (p<0.001). The magnitudes of the decrease in transepidermal water loss from the face and forearm were significantly larger in the probiotic group compared with the controls at weeks 4 and 12 (face) and weeks 8 and 12 (forearm).

Facial wrinkles were measured using a 3D skin imaging system (PRIMOS Premium; GFMesstechnik GmbH, Teltow, Germany) that makes optical 3D measurements based on digital stripe projections using digital micromirror device technology. This system permits a quantitative analysis of wrinkles at the skin surface. Skin wrinkles were measured at the outer corners of the left or right eye and analyzed in terms of the parameters.

The results showed improvement from baseline in all wrinkle parameters (Ra, Rmax, Rp, Rv, and Rz) in both groups. By week 12, decreases in the parameters were 43.48% (Ra), 65.22% (Rmax), 7.80% (Rp), 106.82% (Rv), and 30.75% (Rz). Decreases in Ra, Rmax, and Rv were significantly larger (Ra, Rmax, p<0.05; Rv, p<0.01) in the probiotic group vs. the placebo group at week 12. Representative digital and 3D images of facial skin following week 12.

Facial skin gloss was measured by a glossmeter (Delfin Technologies, Kuopio, Finland). This device consists of a 635 nm red semiconductor diode laser and a mirror in a chamber. When the chamber is placed on the skin, skin gloss is measured by the degree of specular or light scattering. Skin gloss was measured three times at the left or right cheek at the point where a vertical line from the pupil meets a horizontal line from the end of the nose, and the average values of the measurements were used in the analysis.

A proprietary device called the GlossMeter was used to evaluate the effect of Lactobacillus Plantarum formulation on skin gloss. The result showed that skin gloss improved in both groups during the study period, but that the rates of improvement were much better (16.54%, p<0.05) in the probiotic group by week 12.

Skin elasticity was measured by using a cutometer (MPA580; Courage and Khazaka Electronic GmbH). The measuring principle is based on suction and elongation. The device generates 450 mbar of negative pressure, and the skin is drawn into the aperture of the probe under constant negative pressure for 2 sec (on-time). The negative pressure is switched off for 2 sec to allow the skin to return to its original shape (off-time). Each measuring cycle consists of three repetitions of on-time/off-time. Skin elasticity was measured at the left or right cheek using the point marked by the intersection of a vertical line from the outer corner of the eye and the horizontal line from the tip of the nose and the R2 value (gross elasticity; Ua/Uf) was analyzed.

Skin elasticity increased gradually in both groups during the study period, but the degree of improvement at week 4 (13.17%, p<0.05) and week 12 (21.73%; p<0.01) was significantly higher in the probiotic group compared with the placebo group.

All statistical analyses were performed using the SPSS Package Program (IBM, USA). Normality of the distribution of data was assessed using kurtosis and skewness, and prior homogeneity was analyzed using an independent t-test. The statistical significance of the differences between two groups was determined using repeated measures ANOVA. A p-value of <0.05 was considered statistically significant.

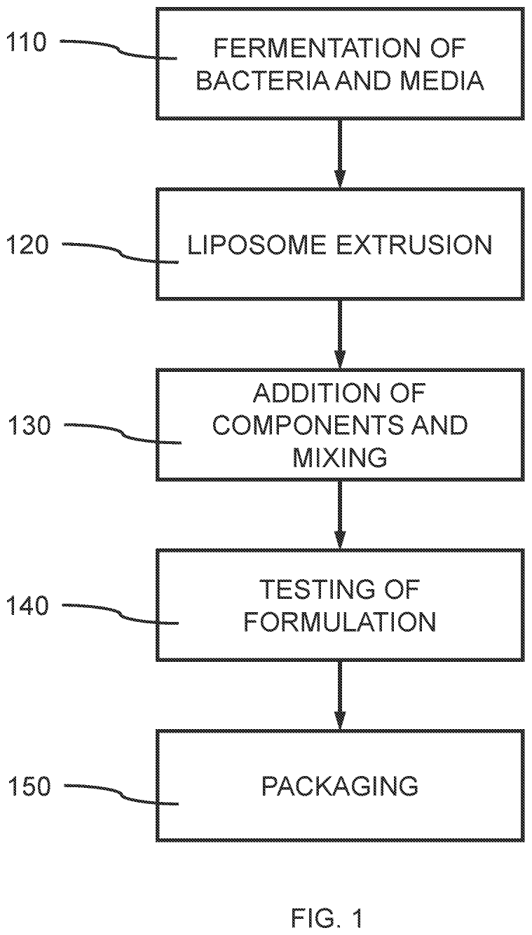

FIG. 1 shows a block/flow diagram for a process of preparing the liposomal formulation including the Lactobacillus plantarum according to an embodiment of the invention.

At block 110, the Lactobacillus Plantarum can be fermented and grown in the fermentation media.

At block 120, the grown Lactobacillus Plantarum can be mixed with the topical formulation ingredients and extruded.

At block 130, the additional components can be added to the extrusion product.

At block 140, the topical formulation can be tested for particle size and quality control.

At block 150, the topical formulation including the probiotic ingredient as formulated according to the present invention can be suitably packaged.

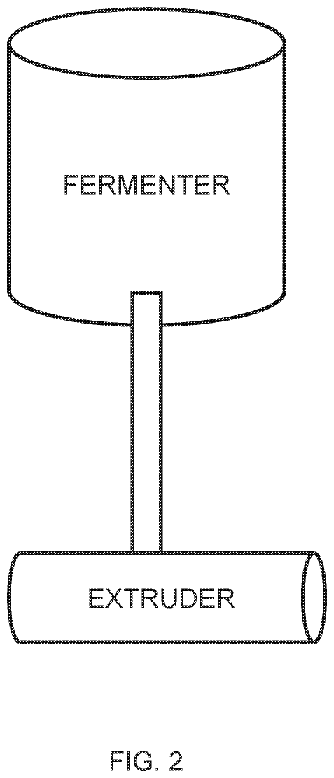

FIG. 2 shows an illustration of the fermenter and extruder portions of the process according to an embodiment of the invention.

In various embodiments, the Lactobacillus Plantarum can be fermented and grown in the fermentation media in a stirred tank at a controlled temperature.

The Lactobacillus Plantarum fermentation media can be transferred to a microfluidizer extruder to be mixed with the formulation ingredients and form the liposomes.

While there have been shown, described and pointed out fundamental novel features of the present principles and embodiments, it will be understood that various omissions, substitutions and changes in the form and details of the described embodiments may be made by those skilled in the art without departing from the spirit of the invention. For example, it is expressly intended that all combinations of those elements and/or method steps which perform substantially the same function in substantially the same way to achieve the same results are within the scope of the present invention. Moreover, it should be recognized that structures and/or features and/or method steps shown and/or described in connection with any disclosed form or implementation of the present principles and embodiments may be incorporated in any other disclosed, described or suggested form or implementation. It is the intention, therefore, to be limited only as indicated by the scope of the claims appended hereto.

It should also be understood that the example embodiments disclosed and taught herein are susceptible to numerous and various modifications and alternative forms. Thus, the use of a singular term, such as, but not limited to, “a” and the like, is not intended as limiting of the number of items. Furthermore, the naming conventions for the various components, functions, parameters, thresholds, and other elements used herein are provided as examples, and can be given a different name or label. The use of the term “or” is not limited to exclusive “or” but can also mean “and/or”.

1. A liposomal vegan probiotic formulation comprising:
a fermented Lactobacillus in an amount in a range of 1×10⁹ to 1×10¹¹ CFU/g;
a polyol in an amount in a range of 1% to 20 wt. %;
a non-ionic surfactant in an amount in a range of 0.005% to 0.1 wt. %;
a phospholipid in an amount in a range of 0.000002% to 0.00004 wt. %; and
an oil making up a balance of the formulation weight.

2. A liposomal vegan probiotic formulation comprising:
a fermented Lactobacillus in an amount in a range of about 1×10⁹ to 1×10¹¹ CFU/g;
propanediol in an amount in a range of 1% to 20 wt. %;
1,2-hexanediol in an amount in a range of 0.1% to 2 wt. %;
ethyl-hexyl-glycerin in an amount in a range of 0.005% to 0.1 wt. %;
caprylic/capric triglyceride in an amount in a range of 0.000002% to 0.00004 wt. %;
hydrogenated lecithin in an amount in a range of 0.000001% to 0.00002 wt. %;
polyglyceryl-6 caprylate in an amount in a range of 0.0000005% to 0.000001 wt. %; and
polyglyceryl-4 caprate in an amount in a range of 0.0000005% to 0.000001 wt. %. with a balance water (H₂O).

3. The liposomal vegan probiotic formulation of claim 2, wherein the formulation is encapsulated in a liposome.

4. A method of regulating skin hydration in a human subject comprising:
topically administering to the human subject a skin formulation including an amount of fermented Lactobacillus in a range of 1×10⁹ to 1×10¹¹ CFU/g.

5. The method of claim 4, wherein the formulation further comprises:
propanediol in an amount in a range of about 1 wt. % to about 20 wt. %;
1,2-hexanediol in an amount in a range of about 0.1 wt. % to about 2 wt. %;
ethyl-hexyl-glycerin in an amount in a range of about 0.005 wt. % to about 0.1 wt. %;
caprylic/capric triglyceride in an amount in a range of about 0.000002 wt. % to about 0.00004 wt. %;
hydrogenated lecithin in an amount in a range of about 0.000001 wt. % to about 0.00002 wt. %;
polyglyceryl-6 caprylate in an amount in a range of about 0.0000005 wt. % to about 0.000001 wt. %; and
polyglyceryl-4 caprate in an amount in a range of about 0.0000005 wt. % to about 0.000001 wt. %.

6. The method of claim 5, wherein the formulation is applied topically for a period of at least 12 weeks.

7. A method of preparing a liposomal vegan probiotic formulation comprising:
fermenting a Lactobacillus media composition;
extracting a liposome mixture from the fermented Lactobacillus media composition;
adding propanediol in an amount in a range of 1% to 20%;
adding 1,2-hexanediol in an amount in a range of 0.1% to 2%;
adding ethyl-hexyl-glycerin in an amount in a range of 0.005% to 0.1%;
adding caprylic/capric triglyceride in an amount in a range of 0.000002% to 0.00004%;
adding hydrogenated lecithin in an amount in a range of 0.000001% to 0.00002%;
adding polyglyceryl-6 caprylate in an amount in a range of 0.0000005% to 0.000001%;
adding polyglyceryl-4 caprate in an amount in a range of 0.0000005% to 0.000001%.
heating the mixture to 80C to 90C; and
extruding the mixture at 300 rpm to 600 rpm in 5 minutes to 20 min using a microfluidizer at a pressure in a range of about 500 bar to about 1500 bar.

8. The method of claim 7, further comprising, testing the extruded liposome mixture to determine a particle size having diameter values in a range of about 100 nanometers to about 300 nm.
